(19) United States
(12) Patent Application Publication
(10) Pub. No.: US 2025/0336480 A1
(43) Pub. Date: Oct. 30, 2025

(54) MOLECULAR FILE SYSTEM

(71) Applicant: North Carolina Agricultural and Technical State University, Greensboro, NC (US)

(72) Inventors: Reza Zadegan, Greensboro, NC (US); Jorge Eduardo Guerrero, Coral Springs, FL (US)

(21) Appl. No.: 19/189,507

(22) Filed: Apr. 25, 2025

Related U.S. Application Data
(60) Provisional application No. 63/638,808, filed on Apr. 25, 2024.

(51) Int. Cl. G16B 50/30; G16B 30/00

(52) U.S. Cl. CPC G16B 50/30; G16B 30/00

(57) ABSTRACT

A system includes one or more processors that perform operations comprising: receiving, during a first session, a first input corresponding to a first modification of an object stored in a molecular data storage-based memory device; generating, during the first session, a first data block based on the first input and one or more previous data blocks generated during a previous session that precedes the first session; generating, and during the first session, a first index block that corresponds to the first data block and comprises first metadata corresponding to the first input; encoding, during the first session, the first data block and the first index block into a first DNA sequence; and storing, during the first session, the first DNA sequence in a DNA pool of the molecular data storage-based memory device.

### CLAIM OF PRIORITY

This application claims priority from U.S. Provisional Patent Application Ser. No. 63/638,808 entitled “Molecular File System,” filed Apr. 25, 2024, with the United States Patent and Trademark Office, the disclosure of which is incorporated by reference herein in its entirety.

### STATEMENT AS TO RIGHTS TO INVENTIONS MADE UNDER FEDERALLY SPONSORED RESEARCH OR DEVELOPMENT

This invention was made with Government support under 2027738 awarded by the National Science Foundation. The Government has certain rights in this invention.

### TECHNICAL FIELD

The present disclosure relates generally to a molecule-based data storage that enables storing and organizing files. A Molecular File System (MolFS) disclosed herein includes a protocol and data structure that guide the operating system in storing and retrieving digital data from molecular data storage-based devices.

### BACKGROUND

Scaling, automation, and energy consumption during information storage, processing, and transmission are some challenges of the semiconductors and information technology industries. While semiconductors' performance and capacity increase and their energy consumption and cost decrease continuously, the industry faces significant challenges, including capacity limitation and tremendous environmental footprint. Alternative technologies include molecular data storage-based memory devices, such as a DNA-based memory (e.g., a memory device that converts digital data into a binary code and encodes the binary code into synthesized strands of DNA). The features of a DNA-based memory, including improved security, storage density, energy consumption, error tolerance, longevity, and stability compared to other archival data storage mediums, make it an intriguing candidate for data storage. Moreover, recent advancements in DNA synthesis and sequencing technologies have enabled the feasible writing and reading of digital data into DNA sequences.

### SUMMARY

The present disclosure provides a system comprising one or more processors and one or more nontransitory computer-readable mediums comprising instructions that, when executed by the one or more processors, cause the one or more processors to perform operations comprising: receiving, by the one or more processors and during a first session, a first input corresponding to a first modification of an object stored in a molecular data storage-based memory device; generating, by the one or more processors and during the first session, a first data block based on the first input and one or more previous data blocks generated during a previous session that precedes the first session; generating, by the one or more processors and during the first session, a first index block that corresponds to the first data block and comprises first metadata corresponding to the first input; encoding, by the one or more processors and during the first session, the first data block and the first index block into a first DNA sequence; and storing, by the one or more processors and during the first session, the first DNA sequence in a DNA pool of the molecular data storage-based memory device.

In some embodiments, the system recited in the above paragraph may include or may perform operations comprising: generating the first data block based on the first input and the one or more previous data blocks further comprises performing a Myers difference routine based on the first input and the one or more previous data blocks; the operations further comprise decoding, by the one or more processors and during a second session that is after the first session, the first DNA sequence to obtain the first data block, the first index block, the one or more previous data blocks, and one or more previous index blocks that respectively correspond to the one or more previous data blocks; decoding the first DNA sequence further comprises performing a generic sequencing routine; the operations further comprise displaying, by the one or more processors and during the second session, user interface elements corresponding to the first data block and the one or more previous data blocks; the operations further comprise: receiving, by the one or more processors and during the second session, a second input corresponding to a second modification of the object; generating, by the one or more processors and during the second session, a second data block based on the second input, the first data block, and the one or more previous data blocks; generating, by the one or more processors and during the second session, a second index block that corresponds to the second data block and comprises second metadata corresponding to the second input; encoding, by the one or more processors and during the second session, the first data block, the first index block, the one or more previous data blocks, the one or more previous index blocks, the second data block, and the second index block into a second DNA sequence; and storing, by the one or more processors and during the second session, the second DNA sequence in the DNA pool of the molecular data storage-based memory device; the first data block comprises binary data segments that correspond to the first modification of the object, and wherein the one or more previous data blocks comprise binary data segments that respectively correspond to one or more previous modifications of the object; encoding the first data block and the first index block into the first DNA sequence further comprises encoding the binary data segments and the first metadata based on a sequential nucleic acid memory (SeqNAM) routine or any other encoding and decoding algorithm known in the art; and/or the object is one of a file or a folder.

The present disclosure provides a method of operating a molecular data storage-based memory device, the method including: receiving, by one or more processors and during a first session, a first input corresponding to a first modification of an object stored in the molecular data storage-based memory device, where the object is one of a file or a folder; performing, by the one or more processors and during the first session, a Myers difference routine to generate a first data block based on the first input and one or more previous data blocks generated during a previous session that precedes the first session; generating, by the one or more processors and during the first session, a first index block that corresponds to the first data block and comprises first metadata corresponding to the first input; encoding, by the one or more processors and during the first session, the first data block and the first index block into a first DNA sequence; and storing, by the one or more processors and during the first session, the first DNA sequence in a DNA pool of the molecular data storage-based memory device.

In some embodiments, the method recited in the above paragraph may include: decoding, by the one or more processors and during a second session that is after the first session, the first DNA sequence to obtain the first data block, the first index block, the one or more previous data blocks, and one or more previous index blocks that respectively correspond to the one or more previous data blocks; decoding the first DNA sequence further comprises performing a generic sequencing routine; displaying, by the one or more processors and during the second session, user interface elements corresponding to the first data block and the one or more previous data blocks; receiving, by the one or more processors and during the second session, a second input corresponding to a second modification of the object; generating, by the one or more processors and during the second session, a second data block based on the second input, the first data block, and the one or more previous data blocks; generating, by the one or more processors and during the second session, a second index block that corresponds to the second data block and comprises second metadata corresponding to the second input; encoding, by the one or more processors and during the second session, the first data block, the first index block, the one or more previous data blocks, the one or more previous index blocks, the second data block, and the second index block into a second DNA sequence; and storing, by the one or more processors and during the second session, the second DNA sequence in the DNA pool of the molecular data storage-based memory device; the first data block comprises binary data segments that correspond to the first modification of the object, and wherein the one or more previous data blocks comprise binary data segments that respectively correspond to one or more previous modifications of the object; and encoding the first data block and the first index block into the first DNA sequence further comprises encoding the binary data segments and the first metadata based on a sequential nucleic acid memory (SeqNAM) or any other encoding and decoding algorithm known in the art.

The present disclosure provides a system comprising one or more processors and one or more nontransitory computer-readable mediums comprising instructions that, when executed by the one or more processors, cause the one or more processors to perform operations comprising: receiving, by the one or more processors and during a first session, a first input corresponding to a first modification of an object stored in a molecular data storage-based memory device, where the object is one of a file or a folder; performing, by the one or more processors and during the first session, a Myers difference routine to generate a first data block based on the first input and one or more previous data blocks generated during a previous session that precedes the first session; generating, by the one or more processors and during the first session, a first index block that corresponds to the first data block and comprises first metadata corresponding to the first input; encoding, by the one or more processors and during the first session, the first data block and the first index block into a first DNA sequence; storing, by the one or more processors and during the first session, the first DNA sequence in a DNA pool of the molecular data storage-based memory device; and decoding, by the one or more processors and during a second session that is after the first session, the first DNA sequence to obtain the first data block, the first index block, the one or more previous data blocks, and one or more previous index blocks that respectively correspond to the one or more previous data blocks.

These and other embodiments are described in greater detail in the detailed description which follows. An object of the presently disclosed subject matter having been stated hereinabove, and which is achieved in whole or in part by the presently disclosed subject matter, other objects will become evident as the description proceeds when taken in connection with the accompanying drawings as best described herein below. It is intended that all such additional embodiments, in addition to any and all combinations of the above embodiments, be included within this description, be within the scope of the present disclosure, and be protected by the accompanying claims.

### BRIEF DESCRIPTION OF THE DRAWINGS

FIG. 1A is a block diagram of an example MolFS according to some embodiments of the present disclosure.

FIG. 1B is a block diagram illustrating an example operation of the MolFS according to some embodiments of the present disclosure.

FIG. 2 is a block diagram illustrating a plurality of sessions of the MolFS according to some embodiments of the present disclosure.

FIG. 3 is a block diagram illustrating an example save session and a restore session of the MolFS according to some embodiments of the present disclosure.

FIG. 4 is a diagram illustrating an example a file restoration routine performed by the MolFS according to some embodiments of the present disclosure.

FIG. 5A is a graph illustrating one or more performance characteristics of the MolFS according to some embodiments of the present disclosure.

FIGS. 5B and 5C are tables illustrating file sizes and oligonucleotide (oligo) requirements, respectively, of the MolFS according to some embodiments of the present disclosure.

FIGS. 5D and 5E are tables illustrating simulation data of the MolFS according to some embodiments of the present disclosure.

FIG. 5F is a graph illustrating one or more performance characteristics of the MolFS according to some embodiments of the present disclosure.

FIGS. 6A, 6B, 6C, 6D, and 6E are graphs illustrating one or more performance characteristics of the MolFS according to some embodiments of the present disclosure.

### DETAILED DESCRIPTION

The presently disclosed subject matter will now be described more fully. The presently disclosed subject matter can, however, be embodied in different forms and should not be construed as limited to the embodiments set forth herein below and in the accompanying Examples. Rather, these embodiments are provided so that this disclosure will be thorough and complete, and will fully convey the scope of the embodiments to those skilled in the art.

All references listed herein, including but not limited to all patents, patent applications and publications thereof, and scientific journal articles, are incorporated herein by reference in their entireties to the extent that they supplement, explain, provide a background for, or teach methodology, techniques, and/or compositions employed herein.

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs.

The terms “a”, “an”, and “the” refer to “one or more” when used in this application, including the claims.

The term “and/or” when used in describing two or more items or conditions, refers to situations where all named items or conditions are present or applicable, or to situations wherein only one (or less than all) of the items or conditions is present or applicable.

The use of the term “or” in the claims is used to mean “and/or” unless explicitly indicated to refer to alternatives only or the alternatives are mutually exclusive, although the disclosure supports a definition that refers to only alternatives and “and/or.”

As used herein “another” can mean at least a second or more.

The term “comprising”, which is synonymous with “including,” “containing,” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed subject matter can include the use of either of the other two terms.

As used herein, the term “about”, when referring to a value is meant to encompass variations of in one example ±20% or ±10%, in another example ±5%, in another example ±1%, and in still another example ±0.1% from the specified amount, as such variations are appropriate to perform the disclosed methods.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

All ranges disclosed herein are also to be considered to include the end points of the range, unless expressly stated otherwise. For example, a range of “between 5 and 10”, “from 5 to 10” or “5-10” should generally be considered to include the end points 5 and 10.

Further, when the phrase “up to” is used in connection with an amount or quantity, it is to be understood that the amount is at least a detectable amount or quantity. For example, a material present in an amount “up to” a specified amount can be present from a detectable amount and up to and including the specified amount.

In this application, the term “controller” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit (e.g., one or more processors); other suitable hardware components that provide the described functionality, such as, but not limited to, transceivers, routers, input/output interface hardware, among others; or a combination of some or all of the above, such as in a system-on-chip. The term “code,” as used herein, may include software, firmware, and/or microcode, and may refer to computer programs, routines, functions, classes, data structures, and/or objects. The computer programs may include: (i) descriptive text to be parsed, (ii) assembly code, (iii) object code generated from source code by a compiler, (iv) source code for execution by an interpreter, (v) source code for compilation and execution by a just-in-time compiler, etc. The term “memory” is a subset of the term computer-readable medium. The term “nontransitory computer-readable medium,” as used herein, does not encompass transitory electrical or electromagnetic signals propagating through a medium (such as on a carrier wave). Non-limiting examples of a non-transitory, tangible computer-readable medium are nonvolatile memory circuits, such as a flash memory circuit, an erasable programmable read-only memory circuit, or a mask read-only circuit, and volatile memory circuits, such as a static random access memory circuit or a dynamic random access memory circuit.

The present disclosure has been described herein with reference to flowchart and/or block diagram illustrations of methods, systems, and devices in accordance with example embodiments of the present disclosure. It will be understood that each block of the flowchart and/or block diagram illustrations, and combinations of blocks in the flowchart and/or block diagram illustrations, may be implemented by computer program instructions and/or hardware operations. These computer program instructions may be provided to a processor of a general purpose computer, a special purpose computer, or other programmable data processing apparatus to produce a machine, such that the instructions, which execute via the processor of the computer or other programmable data processing apparatus, are configured to implement the functions specified in the flowchart and/or block diagram block or blocks.

These computer program instructions may also be stored in a non-transitory computer usable or computer-readable memory that may direct a computer or other programmable data processing apparatus to function in a particular manner, such that the instructions stored in the computer usable or computer-readable memory produce an article of manufacture including instructions that implement the function specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions that execute on the computer or other programmable apparatus provide steps for implementing the functions specified in the flowchart and/or block diagram block or blocks.

Computer data storage technology utilizes various physical properties, including magnetic fields (in hard disks and tapes), electrical charges (in flash drives), and optical diffraction (in CDs and DVDs) to store and organize data. The raw binary data of the device is formatted into a file system, such as NTFS, FAT, or UDF. Each device defines physical locations for sectors and blocks, where a block is the minimal storage unit that stores a fixed number of bits. The operating system reads an index file, typically located in the first block of the device, which contains the file structure and the physical location of the files across the logical blocks of the storage device. The user interacts with the operating system to create folders and to add and edit files. The file system stores the binary contents in the blocks and updates the file structure in the index file. Unlike current digital data storage media, a DNA memory device does not have a fixed physical structure or a controlling method that locates various regions of stored data.

Compared to silicon-based technologies, a DNA memory device has higher bit density, longer data retention, lower maintenance energy usage, and less environmental footprint. Various approaches demonstrate the capabilities of DNA memory devices for data storage, including noise-tolerance, random access, and automation. Enabling on-demand editing is a desirable feature of a data storage system, while data editing remains a significant challenge in practical DNA data storage. A few bits have been modified by replacing segments of DNA strands enabling and disabling nicking places in DNA backbone, replacing overhangs, or mutating nucleotides. Modifying and accessing specific nucleotide sequences in a pool of DNA strands is extremely tedious, and while it is possible to use laborious methods to access, pull out, and replace DNA oligos (e.g., using specific primers), such methods may not be scalable and readily used in a practical storage device.

In silicon-based memory devices, modifying the digital contents of a file may require altering several non-consecutive bits. In addition, if the encoding scheme contains error correction codes, the modification procedure may consider all the parity bits to maintain compatibility with the error-correction code; otherwise, it may be detected as bit corruption. Computer data storage technology utilizes various physical properties, including magnetic fields (in hard disks and tapes), electrical charges (in flash drives), and optical diffraction (in CDs and DVDs) to store and organize data. The raw binary data of the device is formatted into a file system, such as NTFS, FAT, or UDF. Each device defines physical locations for sectors and blocks, where a block is the minimal storage unit that stores a fixed number of bits. The operating system reads an index file, typically located in the first block of the device, which contains the file structure and the physical location of the files across the logical blocks of the storage device. The user interacts with the operating system to create folders and to add and edit files, and the file system stores the binary contents into the blocks and updates the file structure into the index file.

Some DNA memory specifications aim to enable archive readers to find the molecular data storage-based data storage systems' initial booting sequences. Two example specifications include, but are not limited to: i) Sector Zero, which defines the minimal amount of information needed for the archive reader to identify the coder/decoder (CODEC) for the data in the next sector and the source, and ii) Sector One, which includes information such as a description of contents, a file table, and parameters to transfer to a sequencer. These specifications aim to guide the engaged companies' adaptation and implementation of standard data management. In March 2024, DNA data storage alliance (SNIA) affiliate developed the aforementioned CODEC. SNIA is an Industry organization that develops global standards for data related technology.

Among computer data storage technologies, CD-R and Tape File Systems allow the file overwrite action by performing an append-only strategy. CD-R utilizes the UDF (Universal Disk Format) to incorporate multisession features. In contrast, Tape employs the LTFS (Linear Tape File System) to optimize sequential access by appending blocks to organize the binary contents. Both schemes handle the devices as write-once, read, and append-only, which is similar to the characteristics of DNA storage.

Disclosed herein is a protocol that enables the organization, storage, and editing of molecular and molecular data storage-based digital memory (e.g., a DNA-based storage device), employing an append-only strategy to enable practical file editing and organization in DNA data storage. The MolFS described herein incorporates multisession and standard file system functionalities, such as folder creation and file edition.

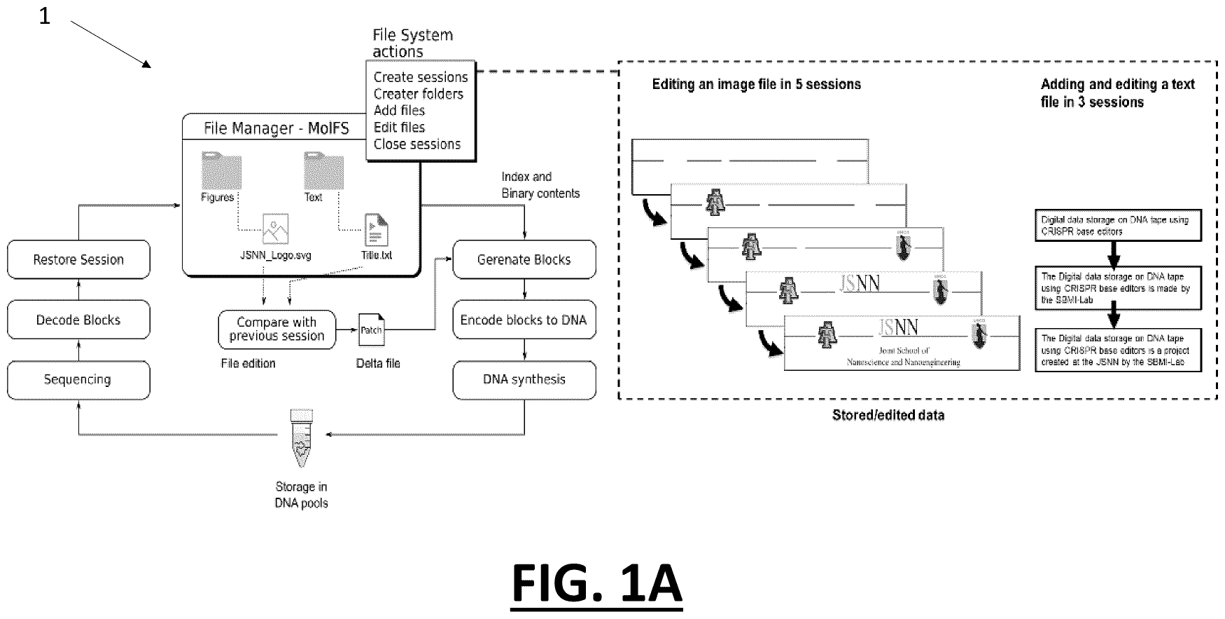

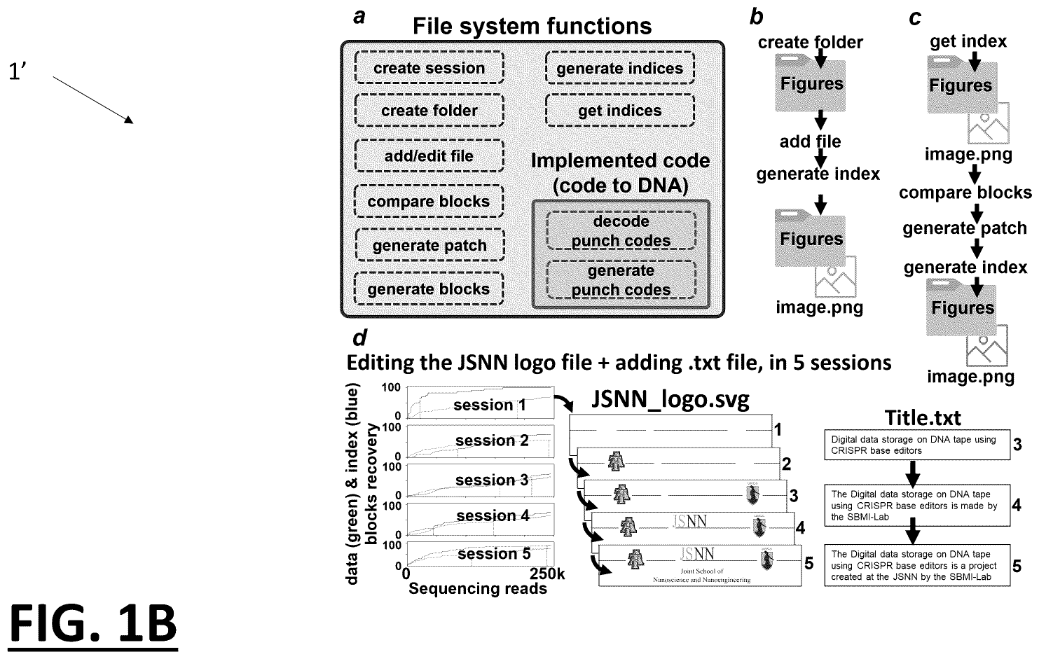

In some embodiments, the MolFS is configured to store and organize files through file systems akin to electronic data storage devices. The MolFS includes a protocol and data structure that guide the operating system in storing and retrieving digital data from molecular data storage-based data storage/memory devices. The file system defines blocks as the primary storage unit; index blocks incorporate metadata of the corresponding session; and data blocks store binary contents of the files. Users may interact with the file system through sessions, where they can create and modify folders and files as desired, as shown by MolFS 1 in FIGS. 1A-1B. Upon completing a session, the user “ejects” the device, prompting the protocol to examine any changes made to the files and collect them into delta files. The filing system generates index and data blocks with the binary contents of the files, including deltas. Then, using a DNA encoding scheme, it transforms the blocks into DNA sequences that will be synthesized into oligos.

Like a removable media, the MolFS 1 enables repeated access to the DNA memory content. To do so, the file system restores the previous sessions. After sequencing the DNA pools, the file system identifies the blocks and restores their raw binary content. For each session, it uses the index blocks to recreate the folder structure and identify the corresponding files. For each file, the index informs the exact location of the binary contents inside the data blocks and rebuilds the files. If the file was modified in the session that is being restored, the file system restores the associated delta file and patches it to the previous version of the file, restoring its contents. Once all the sessions are restored, the file system creates a new session where the user can work with the files. Then the MolFS 1 generates the list of new DNA oligos for the new session that will be added or appended to the existing pools for storage and future data recovery.

An example method performed by the MolFS 1 includes storing, organizing, and editing data through multiple data sessions. To perform the functionality described herein, the MolFS 1 may include one or more processors and one or more nontransitory computer-readable mediums storing instructions that, when executed by the one or more processors, perform the operations described herein.

The user may create, using the MolFS 1, files and folders and may add or modify the files. The developed file system of the MolFS 1 compares the file changes with the previous session to finalize the session, stores the changes in delta (patch) files, and generates the additional blocks for index and data contents. Then, the MolFS 1 utilizes a select encoding scheme to encode the data and generate the lists and sequence contents of the oligo pools. To restore the session, the filing system of the MolFS 1 processes the sequencing data of the DNA pool and retrieves the individual blocks. Finally, the MolFS 1 processes the blocks to restore the previous sessions and prepare the system for a new session workflow. The inset on the right side of FIG. 1A shows an image and a text file in one DNA pool is stored and edited using the MolFS 1.

The file system of the MolFS 1 incorporates overwriting and file system features through the generation of DNA-encoded blocks and the utilization of multisession capabilities. Using a data structure and workflow with an append-only strategy, the MolFS 1 eliminates the need for physical alterations to already stored molecules. File editions are represented in the form of delta files or patches, enabling the retrieval of file states at specific sessions rather than exclusively relying on the last session. To effectively manage the file structure and establish links between individual files and their corresponding patches, the MolFS 1 records metadata in an index file. By storing binary contents in blocks, the file system offers distribution of large files across addressable blocks, which can be stored in one or multiple pools, thus ensuring scalability of the storage system while accommodating the specific requirements of different DNA data storage schemes.

In some embodiments, the MolFS 1 employs DNA blocks to store binary data segments of files. Metadata is stored in Index files, with a new index file created for each session. The index file may indicate the folder hierarchy, stored files, as well as the size and location of these files within the blocks. Upon closing a session, the file system of the MolFS 1 utilizes the Myers' difference (Myers's diff) algorithm to detect file modifications, and then it generates delta files capturing these changes and saves them in new blocks. These delta files are subsequently associated within the index file. During retrieval from the DNA pool, the file system recognizes and restores the index files. For each session, the MolFS 1 compiles the binary files from the blocks and applies the delta files, thereby producing the updated versions of the files.

The file system of the MolFS 1 disclosed herein enables modification of files, where the modifications are transformed into new DNA strands which are added to the existing DNA pool. Additionally, users can create multiple folders and files.

In the file system of the present disclosure, users can start, using the MolFS 1, a session to work with their data and when they are done, they close the session, and the software creates lists and sequences of the new DNA strands for any changes they made.

The MolFS 1 disclosed herein may operate in an “append-only” mode. It may add new DNA strands to modify files, and it may refrain from altering or damaging existing ones. Alternate methods exclude the existing DNA pools that contain old data.

The file system of the MolFS 1 supports the implementation of custom DNA encoding schemes.

The molecular file system of the present application is designed to be compatible with a wide range of molecular storage schemes, and not limited only to DNA. For instance, protein-based data storage can be adapted to use the MolFS 1 of the present disclosure.

As shown in FIG. 1B, which illustrates a block diagram 1′ depicting an example operation of the MolFS 1, the file system uses standard functions to structure, organize, distribute, index, and sectionize the data in data and index blocks. The MolFS 1 may create folders and add files therein, and the file system enables editing by comparing the data sessions and generating the patch (delta) files and new indices. During the data read, the file system uses the patches to instruct the code to apply the appended data. As an example, the MolFS 1 may store and edit the information of a folder, (e.g., an image of a logo “JSNN”), and a text file in DNA data blocks. The original data (1) may be saved in a pool of DNA, and in the subsequent 4 sessions, the MolFS 1 may append the DNA patches to reconstruct the entire logo and add the .txt file.

### EXAMPLE 1

The MolFS 1 may store digital information in DNA pools through multiple sessions. Each session may include index and data blocks. The index blocks may store the file system hierarchy, file locations, and version identifiers. The data blocks may store the digital contents of the files. The system features folder creation and allows users to add and edit files.

Differential files may be used to handle file editions efficiently. The MolFS 1 offers practical and effective storage and editing of digital information in DNA.

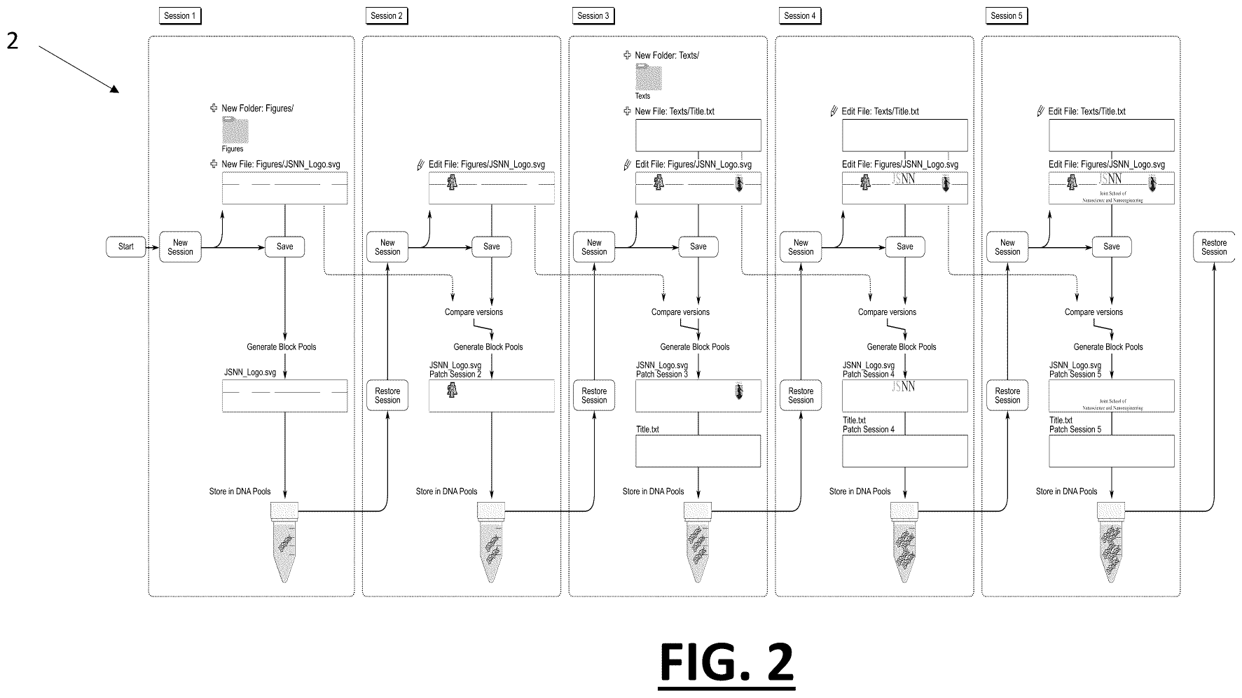

The MolFS 1 uses the Myers' difference algorithm to analyze the differences in the file between sessions and generate a patch file. To restore a session, the MolFS 1 iteratively joins the patches from previous sessions, as shown in sequential session diagram 2 in FIG. 2 showing five iteratively joined sessions.

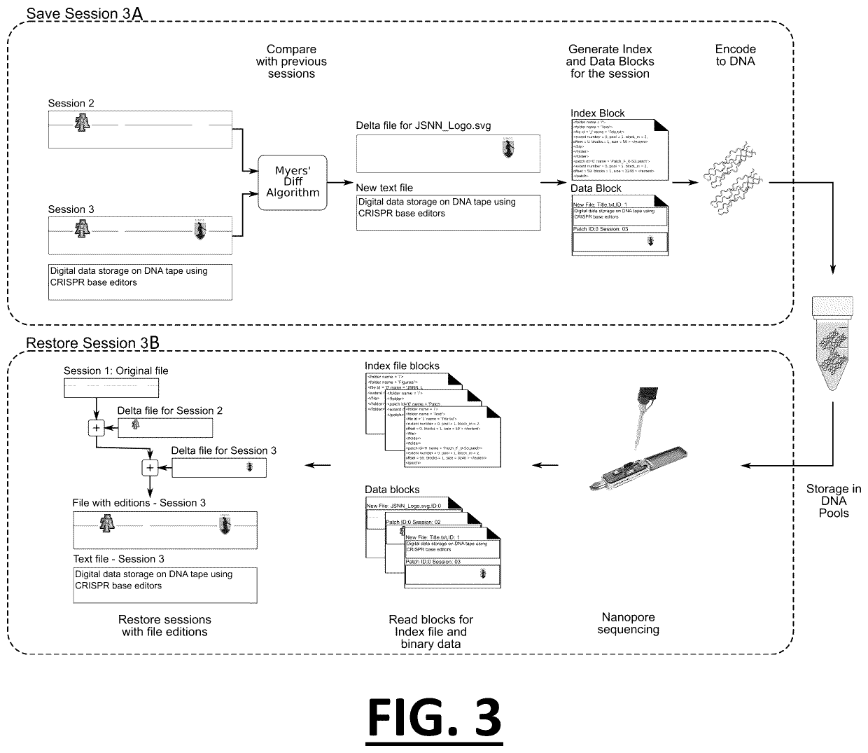

As a specific example and as shown in save session 3A and restore session 3B of FIG. 3A, when saving a session, the MolFS 1 uses the Myers' difference algorithm to compare the changes of each file with respect to the previous session. The file system adds the delta files to the data block of the session and records the relationship between the file and the deltas in the index file. To restore the session from the sequencing data of the DNA pools, the file system of the the MolFS 1 classifies the oligos corresponding to the different blocks and restores the index files. Then, per each session, the file system of the MolFS 1 reconstructs the file structure, retrieves the binary contents of the files from the blocks, and patches the files with the delta files to apply the corresponding modifications.

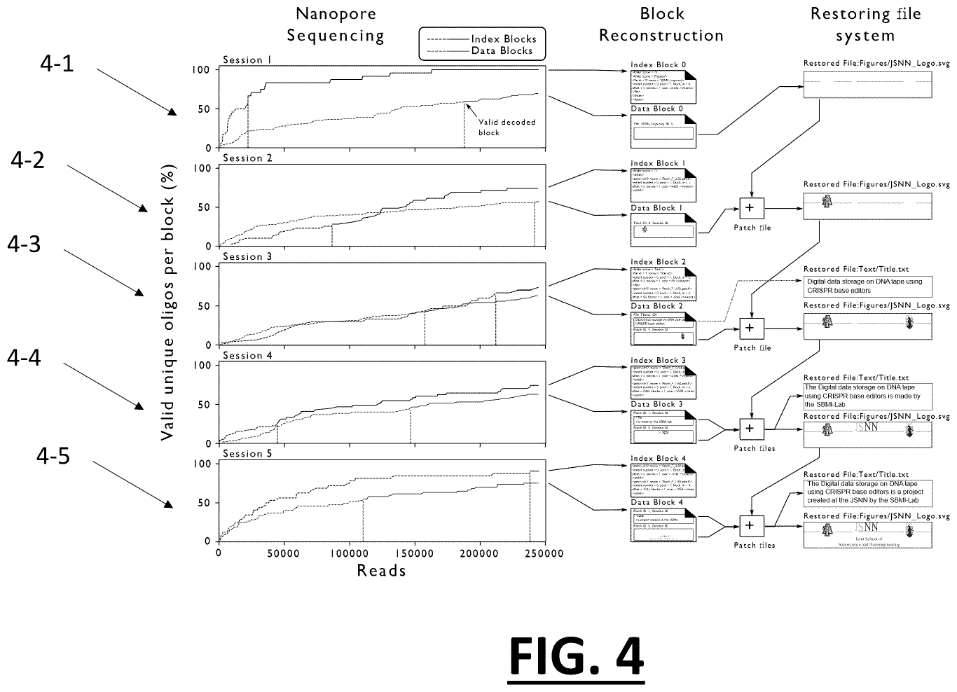

The MolFS 1 disclosed herein is configured to organize and edit files in molecular data storage-based storage. As an example, folders were created and files edited over five sessions 4-1, 4-2, 4-3, 4-4, 4-5 (see FIG. 4), demonstrating its practicality and effectiveness. In the MolFS 1, data blocks were created and automatically encoded into DNA sequences, which were then synthesized and stored in DNA pools. The MolFS 1 retrieved these blocks using generic sequencing (e.g., nanopore sequencing) and iteratively reconstructed the files for each session. These findings demonstrate MolFS is a practical, viable solution for long-term digital information storage in DNA.

In this example, the MolFS 1 includes a protocol and data structure that guide the operating system in storing and retrieving digital data from molecular data storage-based data storage devices. The file system defines blocks as the primary storage unit, whereas index blocks incorporate metadata of the corresponding session, and data blocks store binary contents of the files. Users interact with the filing system of the MolFS 1 through sessions, where they can create and modify folders and files as desired (FIGS. 1A-1B). Upon completing a session, the user “ejects” the device, prompting the protocol to examine any changes made to the files and collect them into delta files. The filing system of the MolFS 1 generates index and data blocks with the binary contents of the files, including deltas. Then, using a DNA encoding scheme, the MolFS 1 transforms the blocks into DNA sequences that will be synthesized into oligos.

Like a removable media, the MolFS 1 enables repeated access to the DNA memory content. To do so, the file system restores the previous sessions. After sequencing the DNA pools, the file system identifies the blocks and restores their raw binary content. For each session, it uses the index blocks to recreate the folder structure and identify the corresponding files. For each file, the index informs the exact location of the binary contents inside the data blocks and rebuilds the files. If the file was modified in the session that is being restored, the file system restores the associated delta file and patches it to the previous version of the file, restoring its contents. Once all the sessions are restored, the file system creates a new session and generates the list of new DNA oligos for the new session that will be added or appended to the existing pools for storage and future data recovery.

### Software Architecture

The MolFS 1 may include controllers (e.g., one or more processors that are configured to execute instructions stored in a non-transitory computer-readable medium) for controlling, generating, manipulating, and/or operating the sessions, indexes, folders, files, extents, and blocks. As an example, a session controller may be used for creating, linking, closing, and restoring sessions. Each session designates its local file structure and associates the root folder with the corresponding Index. The folder controller may collect a hierarchical list of objects, including folder and file objects. The file controller may point to the logical file of the current session and associates its name, a unique identifier number, and the binary extents. Each extent specifies how to locate the binary contents of a file across blocks. The block controller may handle the raw binary data associated with one or multiple files, and the file system allows splitting the binary contents into multiple blocks as needed.

The file system utilizes an interface to enable custom DNA encoding schemes to seamlessly transform binary block contents to and from nucleotide sequences. The file system sends signals through the interface to encode or decode individual blocks, among other functionalities. The file system of the MolFS 1 may employ an interface called SeqNAM (Sequential Nucleic Acid Memory), an encoding scheme based on DNA Fountain encoding. Nonetheless, the MolFS 1 is an open-source platform enabling users to use their independent desired coding schemes as needed. Hence, the spatial density and error-tolerance of the present disclosure may depend on the encoding algorithm, and the density of the data is not necessarily representative of the molecular file systems.

The MolFS 1 incorporates multisession functionality (e.g., FIGS. 2-4), allowing users to open a new session to interact with the file system and close the session when it is ready to write to DNA. The session controller of the MolFS 1 utilizes a version of the Myers' difference algorithm to examine the file changes with respect to previous sessions and generate a delta file. By iteratively patching the files with their delta files, the file system restores the data files with their changes across the sessions, as depicted in FIG. 2.

### Results

To highlight the core capabilities of the MolFS 1, the interface coding scheme (SeqNAM) was first integrated. Next, two folders were created with added and modified vector graphics and text files across five sessions (see FIG. 1A). As a result, the file system of the MolFS 1 generated 10 blocks, which SeqNAM encoded into 614 DNA oligos with an average length of 300 nucleotides. After synthesis and storage, the sequences were retrieved through generic sequencing (e.g., nanopore sequencing), and used the barcodes to classify the oligos to the corresponding blocks and decode their raw binary contents.

FIG. 4 shows the file system's restoration progress after each session. As shown in FIG. 4, five sessions 4-1, 4-2, 4-3, 4-4, 4-5 were stored in DNA oligos, each session comprising the index and data blocks. Using generic sequencing (e.g., nanopore sequencing), the sequences of the oligos were retrieved and each block's binary content recovered through the designated decoding process. With the index and data blocks' data, the MolFS 1 fully restored the files. Across consecutive sessions, the file system patched binary files from previous sessions to update them with the changes made. Each graph 4-1, 4-2, 4-3, 4-4, 4-5 shows the percentage of unique valid oligos associated with each session's Index and Data blocks and the time SeqNAM correctly decodes each block. This encoding allowed the recovery of the blocks while some oligos were not recovered; for instance, the block Index 0 was decoded after 22 k reads with 58% valid oligos. Nonetheless, the recovery of the data may be based at least in part on the interface coding algorithm's (SeqNAM's) capabilities or the capabilities of any other encoding and decoding algorithm known in the art. For the SeqNAM implementation, the decoding parameters determine the number of segments encoded within each block. Without these parameters, SeqNAM decoding becomes an iterative process for guessing the correct number of encoded segments. This can lead to false valid results if the guess is incorrect, especially for larger files. Including the decoding parameters in the index file solves the problem. However, these parameters are not available before reading the blocks containing the index file. To address this issue, the MolFS 1 uses the block flags to detect and prevent false valid results, ensuring accurate decoding.

After decoding the blocks, the file system of the MolFS 1 used the index blocks to restore the file system's structure and retrieve the binary contents of the files from the data blocks. FIGS. 2-4 show the iterative steps for updating the vectorial file for each session until assembling a logo or image file at the fifth session and adding a text file in a different folder from the third session. Each reconstructed session demonstrated that the folders were regenerated with their corresponding hierarchy and name. Additionally, the files contained the desired modifications, demonstrating the overwriting features and file system organization. The edits to replace a data proceed through the same process steps regardless of the content of the file.

Additionally, in-silico simulations demonstrated the advantages of utilizing delta files for storing modifications, compared to replacing entire pools each time, or when sessions are not used. The relative costs of additive file operations may decrease. Conversely, removing file contents increased delta file usage but decreased overall file size. Myers' difference algorithms demonstrate efficiency in generating deltas for text-based files, whereas their effectiveness is lower for binary files, often resulting in larger file sizes. In any of these scenarios, if the delta files exceed the target file's size, the file system of the MolFS 1 omits the delta and regenerates the entire file. Where the proportion of operational overhead is large; it may be due to the small size of the files stored. Simulations indicate the ratio of the overhead depends on the size of the files, and the larger the file, the smaller the proportional overhead costs. Since the majority of the files to be stored in a practical archival storage system are larger than a few kilobytes (KB) stored and edited in the disclosed MolFS 1, the MolFS 1 is practical and can scale well with larger data.

In some embodiments, DNA memory devices are presumed to be a write-once and append-only data storage medium, and hence the file system protocol of the MolFS 1 disclosed herein enables editing of the data as needed. The file system of the MolFS 1 incorporates overwriting and file system features through the generation of DNA-encoded blocks and the utilization of multisession capabilities. Using a data structure and workflow with an append-only strategy, the MolFS 1 eliminates the need for physical alterations to already stored molecules. File editions are represented in the form of delta files or patches, enabling the retrieval of file states at specific sessions rather than exclusively relying on the last session. To effectively manage the file structure and establish links between individual files and their corresponding patches, the MolFS 1 records metadata in an index file. By storing binary contents in blocks, the file system offers a solution for distributing large files across addressable blocks, which can be stored in one or multiple pools. This ensures the scalability of the storage system while accommodating the specific requirements of different DNA data storage schemes.

Custom DNA data storage algorithms can be seamlessly integrated into the MolFS 1 to encode and decode binary block files into DNA pools and vice versa. Currently, designated DNA sequences (called “primers” for their similarity with primer concept in DNA manipulation techniques) are generally employed for addressing and indexing of stored data. Assuming a length of 20 bases per primer, hypothetically, a single DNA pool of double stranded DNA with two primer sets could contain more than 25 billion combinations of data sections. However, there are multiple challenges that make such a strategy unsuitable for a scalable DNA based memory system. For example, the design of primers for the system will be costly since the specific primers should be designed while the encoding is in process to prevent homologies between the primers and the data. Additionally, possible mismatches and inter or intra reactions between primers and other sequences may need to be calculated and avoided. Therefore, these methods are unlikely to satisfy the need of the data storage industry, as they increase the computational cost and reduce the number of possible primer combinations drastically. Solutions for tracking historical data, and existing editing and post write modification techniques are currently limited in scope. The MolFS 1 disclosed herein provides a standard, practical, and scalable solution for data management and editing for DNA based memory. Moreover, the developed protocol is universal, and as such, not only may the users employ any desirable coding scheme to store the data, but they may also utilize the primer combinations as needed. From the user's perspective, the workflow of utilizing the MolFS 1 for storing and editing files resembles the experience of using a USB drive. The MolFS 1 is as an accessible solution for creating practical DNA data storage systems and incorporates features such as file edition, file system structure, scalability and adaptability to custom DNA data storage schemes, and user-friendliness. Additionally, since the data management is developed according to the standards of the memory industry and provide a solution that is independent of DNA sequences it potentially could serve as a platform for any data storage system that use the same principle.

In some embodiments, the MolFS 1 may be implemented using the Python 3 programming language. For tracking the file modifications, “diffutils” was used to generate the delta files. Other tools that deliver similar results may include, but are not limited to, xdelta, git-diff, radiff, and rsync. For the encoding and decoding binary blocks to DNA and vice versa, SeqNAM (Sequential Nucleic Acid Memory), a DNA encoding scheme based on the DNA Fountain method may be employed. The interface for SeqNAM or any other encoding and decoding algorithm known in the art may be written in Python, enabling seamless communication with the MolFS 1. A set of primers may be added to the oligos of the file system interface for SeqNAM to identify the different blocks for Index and data. Accompanied by this interface, a filter step (SeqNAM Filter) may be incorporated that uses alignment tools (e.g., Smith-Waterman algorithm), which identifies the barcodes of the DNA strands, and restores the sequences of the barcodes, and which facilitates the performance of SeqNAM.

### Experimental Setup

For the experimental demonstration, a simplified version of a logo (e.g., the JSNN logo illustrated in FIGS. 1A-1B and 2-4) was created in SVG format. Five versions of the file were prepared by progressively adding the components of the logo until the complete logo in the last version of the file was created. A Python script was used to simulate a user's actions, create the sessions, transfer the example files from the repository to the location of the file system, perform the changes on the files, and close the sessions for further processing the changes and generation of the DNA Pools. Upon generation of the DNA sequences during the experimental setup, DNA oligos were synthesized such that all the DNA strands of the blocks were in the same pool. PCR amplification was performed using the corresponding primers associated with individual DNA blocks. Sequencing was performed using a MinION Mk1B device from Oxford Nanotechnologies. The Guppy base caller was used to generate FastQ files. The MolFS 1 read data from the FastQ files generated after base-calling. A script file provided in the repository may incorporate the steps to gather the FastQ files, filter the sequences, process and classify the strands, and reconstruct the file system.

### Experimental Details

A demonstration experiment comprising five sessions was used to evaluate the functionality of the MolFS 1. The commands described herein were used to configure the following steps. During session 1, the “Pictures” folder was created and added the vector graphics file “JSNN_Logo.svg”. Subsequently, in sessions 2 to 5, the file was modified by adding other parts of a JSNN logo, as illustrated in FIG. 4.

In session 3, the “Texts” folder was created and included the file “Title.txt” in it, which was further modified in sessions 4 and 5. This experimental setup allowed exploration of various configurations, including folder creation (sessions 1 and 3), file addition (sessions 1 and 3), file modification (sessions 2-5), simultaneous file modification and addition (session 3), and modifications of multiple files within a session (sessions 4 and 5).

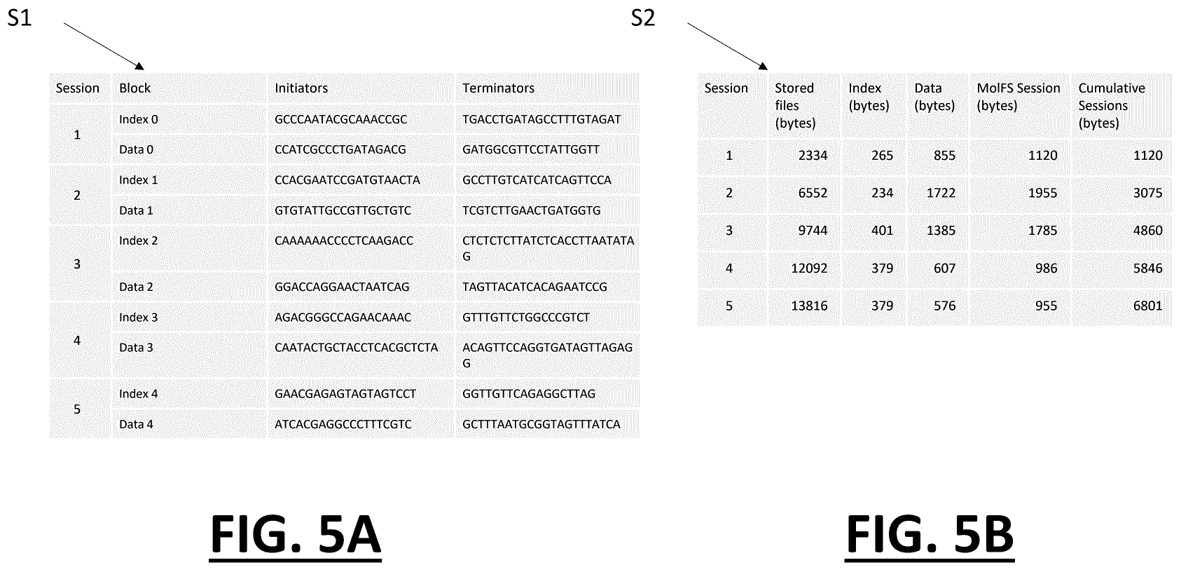

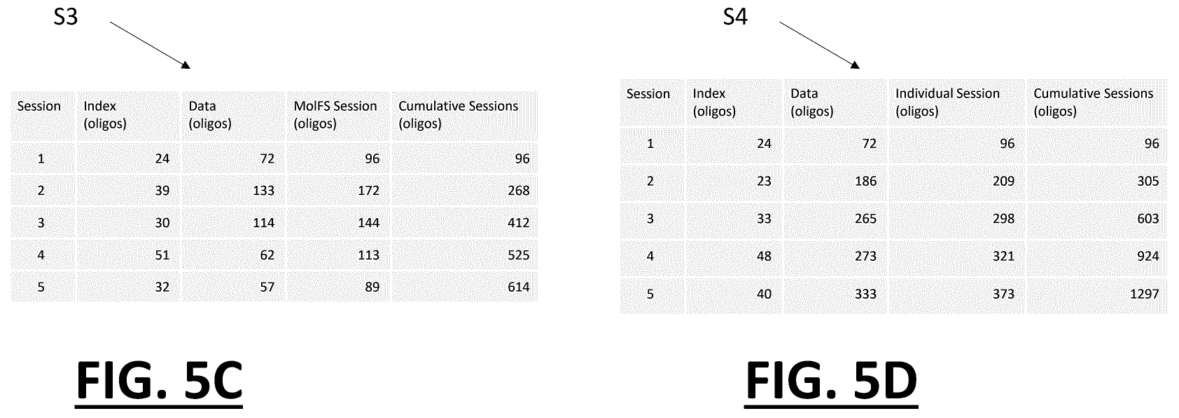

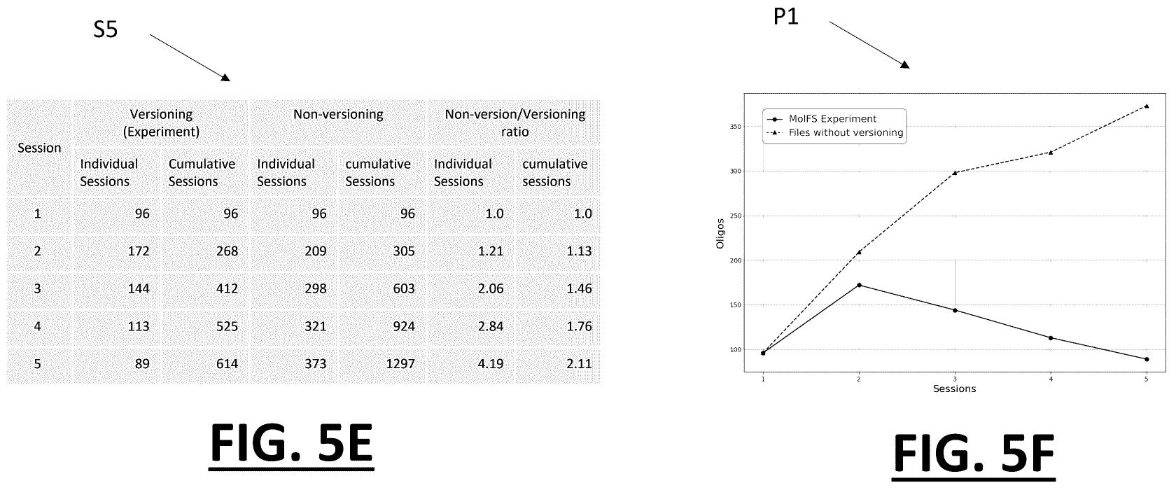

Throughout the experiment, the MolFS 1 generated two blocks for each session, one for the index file and the other for binary data, resulting in a total of 10 blocks. To ensure efficient retrieval, the SeqNAM interface was configured to incorporate barcodes associated with the block identifiers and subsequently encoded the digital contents of the blocks into DNA sequences, listed in Table S1 of FIG. 5A. Table S1 illustrates an interface that adds initiator and terminator sequences to the oligo sequences generated by SeqNAM.

Tables S2 and S3 of FIGS. 5B and 5C, respectively, provide an overview of the file sizes and the number of oligos required to store each session. Notice that with each session, progressively larger graphics files were stored. Table S2 shows that the MolFS 1 session corresponds to the total bytes stored per session, including Index and Data of each session. Table S3 shows that the MolFS 1 saves the binary contents of the blocks in individual files, which SeqNAM transforms into DNA oligos of an average length of 300 nucleotides.

PCR amplification was used per block and two sampling processes to obtain sufficient data. Figure S1 shows the percentage of valid oligos per block from the generic sequencing (e.g., nanopore sequencing) results after basecalling.

The oligo requirements between the MolFS 1 were simulated without versioning, where previous sessions are not linked with new ones. Table S4 of FIG. 5D illustrates the oligo usage for this configuration, and Table S5 of FIG. 5E shows the comparison of MolFS 1 with and without versioning, illustrating the advantages of using versioning.

Table S4 shows the results of using MolFS 1 to create the file system without versioning. That is, the oligos are replaced from scratch for each session. Without versioning, each session does not depend on the other sessions to reconstruct the whole file system at the stage the session represents. Table S5 illustrates the comparison of using MolFS 1 with versioning and without versioning. Using versioning in MolFS 1 implies a reduction in the oligo requirement per session, up to 4.19 times less than not using versioning, as shown by plot P1 of FIG. 5F, but it should be understood that the embodiments are not limited thereto and may include other magnitudes.

### File Size Comparison

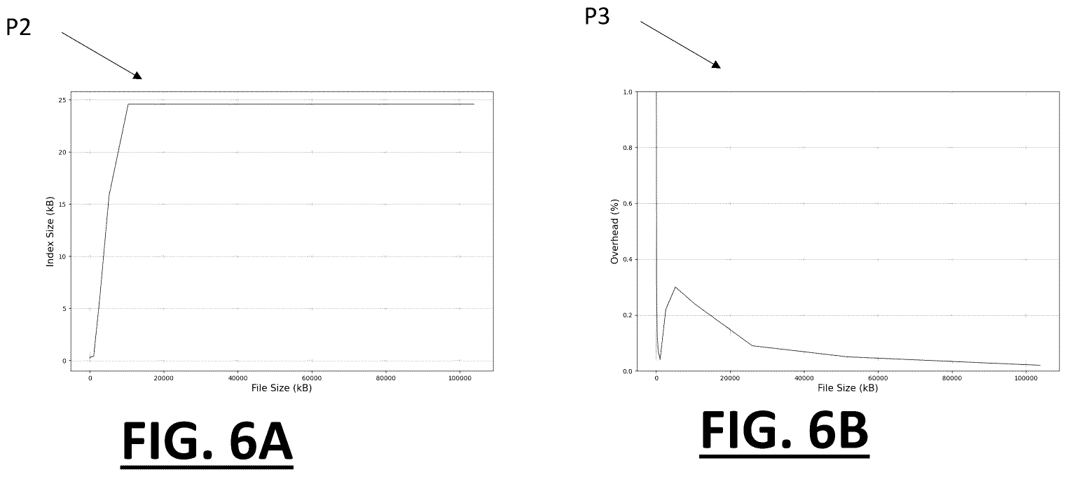

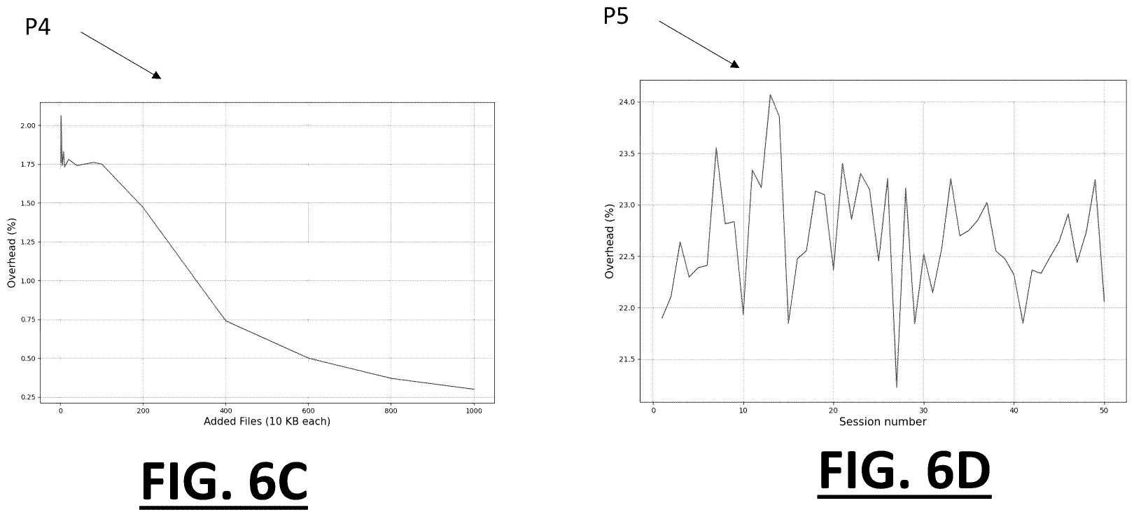

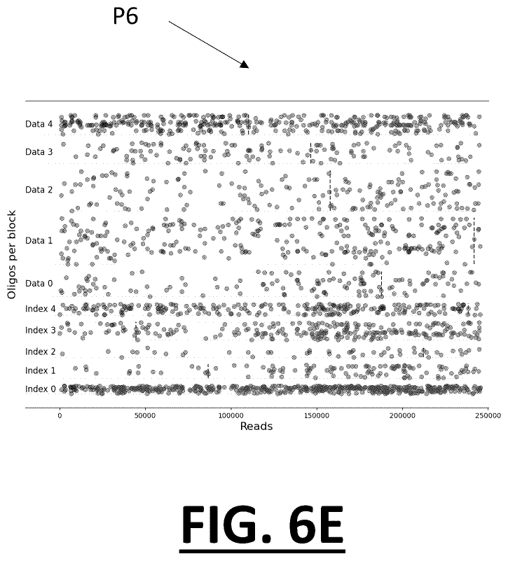

FIGS. 6A-6B show the simulation results of storing a file (from 1 KB to 100 MB of size) using the MolFS 1. The script collected the size of the file (plot P2 of FIG. 6A). Using the Index file, the script calculated the overhead (in percentage) that is shown in plot P3 of FIG. 6B. Note that files bigger than 10 KB need almost the same index size and the larger the file the smaller the cost to file size ratio (overhead).

### Multiple-file Management

Plot P4 of FIG. 6C corresponds to a script that created random files of 10 KB in a single session. Then, the script verified the size of the index file to determine the percentage of the overhead.

### File Edition

Plot P5 of FIG. 6D corresponds to a script that created an initial file of 100 KB, and for each session, it modifies 20% of the content. The cost of modifying a file is: the size of the index file plus the stored delta of the session. The script then sets this value as a percentage of the size of the file.

### Recovery from Generic Sequencing

Plot P6 of FIG. 6E shows the block recovery process from the generic sequencing (e.g., nanopore sequencing) data by showing the percentage of unique recovered oligos per block and marking the read after which the block is correctly decoded. Since all the oligos are contained within the same pool, they were randomly sampled (see plot P1 of FIG. 5F), which is reflected in the FastQ files. The data obtained from generic sequencing (e.g., nanopore sequencing) is processed to retrieve the data and index blocks, which are distributed across a certain number of oligos (indicated by circles). The blue circles of FIG. 6E represent retrieved oligos of the corresponding block before SeqNAM successfully reconstructs the binary or data block. Once the reconstruction is complete, the oligos are represented by green circles of FIG. 6E. The MolFS 1 uses the interface-specific classifications to assign each oligo to the corresponding block, and then the interface (SeqNAM in this case) reconstructs the blocks for both index and data blocks, and the MolFS 1 stores them in a cache folder. Certain essential oligos are either incorrectly decoded or sampled at a later stage, necessitating longer read lengths to ensure complete decoding of the blocks.

As used herein, “molecular device” refers to a molecular data storage system configured to encode digital data to a molecular medium (i.e., a DNA memory device) and vice versa. The MolFS 1 creates individual files representing the binary blocks and uses an interface to communicate with the molecular device to read and write the blocks. In order to properly work as a system, the interface may be capable of addressing individual blocks by their identifier number, as described in detail herein.

As used herein, a “block” is the minimal storage unit used by the MolFS 1, whose size is determined by the encoding technique used by the molecular device. If the size of a file is bigger than the block capacity, the MolFS 1 will generate multiple blocks automatically. The MolFS 1 can incorporate binary segments of multiple files in the same data block if they fit. This feature allows the MolFS 1 to maximize the capacity of the data blocks per session, differing from standard computer systems that do not allow multiple files to share parts of the same data block. The MolFS 1 can compress the binary contents of the blocks before sending them to the molecular device, without affecting the other features of the system. When closing a session, the MolFS 1 encodes the blocks to the corresponding molecular device. If the encoder returns a decoding parameter, the MolFS 1 collects and incorporates it into the Index file, which will be used for further decoding. Once a session is closed, the existing blocks are considered read-only. To add more information to the device, the MolFS 1 will create a new session and its associated data blocks are identified consecutively to the previous ones.

As used herein, “index file” refers to a file that contains information of the file system of the MolFS 1 in an XML-like format. It incorporates the hierarchy of the files per session, file and session metadata and decoding parameters. The index files may also be stored into blocks. By default, the index blocks are stored without compression to increase the chance of decoding using the standard parameters of the molecular device.

As used herein, “folder” refers to a fundamental organizational unit within a file system. It provides a logical structure to store and organize files and other folders. As used herein, “file” refers to a logical entity within the system, assigned with a unique identification number. The binary contents of the file are specified in the extents. By concatenating multiple extents, the complete file is constructed. As used herein, “extent” refers to a binary segment of a file. Depending on its size, it can be stored in one or multiple consecutive blocks.

As used herein, “session” refers to activities made by the user on the File System of the MolFS 1. Each session creates a new index file and data blocks according to the activity. Saving a session involves running a Myers' difference algorithm for each modified file of the session. Reading a session involves reading all the indexes from previous sessions. Restoring an individual file involves reading all the blocks containing segments of the file to retrieve the deltas, and iteratively applying a patching algorithm.

The above-disclosed subject matter is to be considered illustrative, and not restrictive, and the appended claims are intended to cover all such modifications, enhancements, and other embodiments, which fall within the scope of the disclosure. Thus, to the maximum extent allowed by law, the scope is to be determined by the broadest permissible interpretation of the following claims and their equivalents, and shall not be restricted or limited by the foregoing detailed description.

What is claimed is:

1. A system comprising:
one or more processors; and
one or more nontransitory computer-readable mediums comprising instructions that, when executed by the one or more processors, cause the one or more processors to perform operations comprising:
receiving, by the one or more processors and during a first session, a first input corresponding to a first modification of an object stored in a molecular data storage-based memory device;
generating, by the one or more processors and during the first session, a first data block based on the first input and one or more previous data blocks generated during a previous session that precedes the first session;
generating, by the one or more processors and during the first session, a first index block that corresponds to the first data block and comprises first metadata corresponding to the first input;
encoding, by the one or more processors and during the first session, the first data block and the first index block into a first DNA sequence; and
storing, by the one or more processors and during the first session, the first DNA sequence in a DNA pool of the molecular data storage-based memory device.

2. The system of claim 1, wherein generating the first data block based on the first input and the one or more previous data blocks further comprises performing a Myers difference routine based on the first input and the one or more previous data blocks.

3. The system of claim 1, wherein the operations further comprise decoding, by the one or more processors and during a second session that is after the first session, the first DNA sequence to obtain the first data block, the first index block, the one or more previous data blocks, and one or more previous index blocks that respectively correspond to the one or more previous data blocks.

4. The system of claim 3, wherein decoding the first DNA sequence further comprises performing a generic sequencing routine.

5. The system of claim 3, wherein the operations further comprise displaying, by the one or more processors and during the second session, user interface elements corresponding to the first data block and the one or more previous data blocks.

6. The system of claim 3, wherein the operations further comprise:
receiving, by the one or more processors and during the second session, a second input corresponding to a second modification of the object;
generating, by the one or more processors and during the second session, a second data block based on the second input, the first data block, and the one or more previous data blocks;
generating, by the one or more processors and during the second session, a second index block that corresponds to the second data block and comprises second metadata corresponding to the second input;
encoding, by the one or more processors and during the second session, the first data block, the first index block, the one or more previous data blocks, the one or more previous index blocks, the second data block, and the second index block into a second DNA sequence; and
storing, by the one or more processors and during the second session, the second DNA sequence in the DNA pool of the molecular data storage-based memory device.

7. The system of claim 1, wherein the first data block comprises binary data segments that correspond to the first modification of the object, and wherein the one or more previous data blocks comprise binary data segments that respectively correspond to one or more previous modifications of the object.

8. The system of claim 7, wherein encoding the first data block and the first index block into the first DNA sequence further comprises encoding the binary data segments and the first metadata based on a sequential nucleic acid memory (SeqNAM) routine.

9. The system of claim 1, wherein the object is one of a file or a folder.

10. A method of operating a molecular data storage-based memory device, the method comprising:
receiving, by one or more processors and during a first session, a first input corresponding to a first modification of an object stored in the molecular data storage-based memory device, wherein the object is one of a file or a folder;
performing, by the one or more processors and during the first session, a Myers difference routine to generate a first data block based on the first input and one or more previous data blocks generated during a previous session that precedes the first session;
generating, by the one or more processors and during the first session, a first index block that corresponds to the first data block and comprises first metadata corresponding to the first input;
encoding, by the one or more processors and during the first session, the first data block and the first index block into a first DNA sequence; and
storing, by the one or more processors and during the first session, the first DNA sequence in a DNA pool of the molecular data storage-based memory device.

11. The method of claim 10, further comprising decoding, by the one or more processors and during a second session that is after the first session, the first DNA sequence to obtain the first data block, the first index block, the one or more previous data blocks, and one or more previous index blocks that respectively correspond to the one or more previous data blocks.

12. The method of claim 11, wherein decoding the first DNA sequence further comprises performing a generic sequencing routine.

13. The method of claim 11, further comprising displaying, by the one or more processors and during the second session, user interface elements corresponding to the first data block and the one or more previous data blocks.

14. The method of claim 11, further comprising:
receiving, by the one or more processors and during the second session, a second input corresponding to a second modification of the object;
generating, by the one or more processors and during the second session, a second data block based on the second input, the first data block, and the one or more previous data blocks;
generating, by the one or more processors and during the second session, a second index block that corresponds to the second data block and comprises second metadata corresponding to the second input;
encoding, by the one or more processors and during the second session, the first data block, the first index block, the one or more previous data blocks, the one or more previous index blocks, the second data block, and the second index block into a second DNA sequence; and
storing, by the one or more processors and during the second session, the second DNA sequence in the DNA pool of the molecular data storage-based memory device.

15. The method of claim 10, wherein the first data block comprises binary data segments that correspond to the first modification of the object, and wherein the one or more previous data blocks comprise binary data segments that respectively correspond to one or more previous modifications of the object.

16. The method of claim 15, wherein encoding the first data block and the first index block into the first DNA sequence further comprises encoding the binary data segments and the first metadata based on a sequential nucleic acid memory (SeqNAM) routine.

17. A system comprising:
one or more processors; and
one or more nontransitory computer-readable mediums comprising instructions that, when executed by the one or more processors, cause the one or more processors to perform operations comprising:
receiving, by the one or more processors and during a first session, a first input corresponding to a first modification of an object stored in a molecular data storage-based memory device, wherein the object is one of a file or a folder;
performing, by the one or more processors and during the first session, a Myers difference routine to generate a first data block based on the first input and one or more previous data blocks generated during a previous session that precedes the first session;
generating, by the one or more processors and during the first session, a first index block that corresponds to the first data block and comprises first metadata corresponding to the first input;
encoding, by the one or more processors and during the first session, the first data block and the first index block into a first DNA sequence;
storing, by the one or more processors and during the first session, the first DNA sequence in a DNA pool of the molecular data storage-based memory device; and
decoding, by the one or more processors and during a second session that is after the first session, the first DNA sequence to obtain the first data block, the first index block, the one or more previous data blocks, and one or more previous index blocks that respectively correspond to the one or more previous data blocks.

18. The system of claim 17, wherein decoding the first DNA sequence further comprises performing a generic sequencing routine.

19. The system of claim 17, wherein the operations further comprise displaying, by the one or more processors and during the second session, user interface elements corresponding to the first data block and the one or more previous data blocks.

20. The system of claim 17, wherein the operations further comprise:
receiving, by the one or more processors and during the second session, a second input corresponding to a second modification of the object;
generating, by the one or more processors and during the second session, a second data block based on the second input, the first data block, and the one or more previous data blocks;
generating, by the one or more processors and during the second session, a second index block that corresponds to the second data block and comprises second metadata corresponding to the second input;
encoding, by the one or more processors and during the second session, the first data block, the first index block, the one or more previous data blocks, the one or more previous index blocks, the second data block, and the second index block into a second DNA sequence; and
storing, by the one or more processors and during the second session, the second DNA sequence in the DNA pool of the molecular data storage-based memory device.
